(19) United States
(12) Patent Application Publication
(10) Pub. No.: US 2026/0148650 A1
(43) Pub. Date: May 28, 2026

(54) VOCAL PRACTICE APPARATUS, IN PARTICULAR FOR THE STUTTERING TREATMENT

(71) Applicant: VIVAVOCE SRL, Milano (IT)

(72) Inventors: Giovanni Muscara, Milano (IT); Carmine De Vita, Milano (IT)

(21) Appl. No.: 19/121,552

(22) PCT Filed: Oct. 20, 2023
(86) PCT No.: PCT/IB2023/060617

§ 371 Date: Apr. 16, 2025

(30) Foreign Application Priority Data
Oct. 21, 2022 (IT) 102022000021813

(51) Int. Cl. G09B 5/06; A61F 5/58; H04R 1/08; H04R 1/10

(52) U.S. Cl. CPC G09B 5/065; A61F 5/58; H04R 1/08; H04R 1/10; H04R 2420/07

(57) ABSTRACT

A vocal practice apparatus, in particular for the treatment of stuttering; wherein the apparatus comprises: a microphone configured to generate a first audio signal from vocal sounds emitted by a user while performing the exercise; a camera configured to film the user while performing the exercise and to generate a video signal; a first monitor configured to be observed by the user while performing the exercise; a vibrating device configured to transmit a vibratory impulse to the user while performing the exercise; a first headset configured to be worn by the user while performing the exercise; a control unit connected to the microphone, the camera, the first

### CROSS-REFERENCE TO RELATED APPLICATIONS

This Patent Application claims priority from Italian Patent Application No. 102022000021813 filed on Oct. 21, 2022, the entire disclosure of which is incorporated herein by reference.

### TECHNICAL FIELD

The reference technical field of this invention is that of basically electronic apparatuses controlled by special control units that help perform some activities carried out by a user. In particular, this invention relates to an apparatus configured to help a user to carry out vocal exercises aimed at improving speaking skills in terms of quality of elocution or quality of sounds produced. As will be clear, the apparatus of this invention will find advantageous application in the treatment of stuttering.

### STATE OF THE ART

The term “stuttering” refers to an involuntary language disorder characterised by alterations in the rhythm of the word in which language becomes less fluent and difficult due to pauses, repetitions, and extensions of sounds. Stuttering has a prevalence of approximately 1% in the population and an incidence of 4-5%. In Italy, almost one million people stutter with peaks of 5% in the pre-school age equal to approximately 250 thousand children. The statistics indicate, in fact, the second year as the average age of onset. Stuttering manifests itself in different ways that vary from person to person (inter-individual variability). Even in the same subject, stuttering can take on different characteristics in the course of their life (intra-individual variability).

In general, stuttering may be described through some parameters. In particular, these parameters are:
Frequency. Stuttering may only be manifest in rare and specific situations and may not occur in the slightest in all others.
Duration. Stuttering is not always constant. After its manifestation, it may remain totally invisible, silent for some periods, even very long periods, of life. It is also possible that it may reappear suddenly frequently, or sporadically, or more continuously.
Severity. Stuttering is of variable intensity: for example, it may be clearly perceived by the person stuttering but not by those listening or vice versa.
Type. In addition to repetitions, extensions, interruptions, blocks, and circumlocutions, more subtle manifestations exist too. For example, the use of stock phrases (that is) and interjections (well, um), the use of short or broken phrases, the alteration of speech rhythms (speaking more quickly). An intentional increase in the speed of articulation may also represent a strategy for overcoming the block.
Behaviour. Stuttering can also manifest itself through silences or giving up on communicating something, manifestations are often confused with reserve, introversion, or poor communicativeness.

A person who speaks fluently, continuously controls what they say to assess its correctness in terms of production, interrupting themselves if they perceive an error and reformulating the word or phrase correctly. This process of controlling one's language and correcting errors is called speech monitoring. Speech monitoring and the detection of errors, according to the “Perceptual Loop Theory” (Levelt, 1989, 1991; Levelt & Roelofs, 1999), occur in different ways. An internal one, through which language is monitored before being produced. An external one, through which, mainly thanks to hearing, we are able to detect the presence of any errors in what we have said. According to some reliable neuroscientific theories, in people who suffer from stuttering it is plausible that a malfunctioning in this monitoring mechanism is occurring, within the complex process of language production. The concept of speech monitoring is behind one of the most influential models aimed at explaining the motor control of language and the possible anomalies in producing language: the Directions into Velocities of Articulators—DIVA (Guenther, 2006) model. According to this DIVA model, the production of a well learned sound linked to language is obtained through motor controls channelled by two control sub-systems: a “feedforward” sub-system and a “feedback” sub-system. The feedback control subsystem is formed of a hearing component (information linked to sound) and a somatosensory component (information linked to movements of the body, tactile, the position of the muscles and sensory information). The feedforward subsystem is what allows the accurate production of the sounds that characterise language. A child still does not possess accurate motor controls for each sound and it is only through intense practice that it reaches a high level of accuracy in producing the sounds of a given language. This occurs when each control initiated by the feedforward system corresponds to a precise hearing and somatosensory feedback. In fact, the first time that one tries to produce a new sound, one mainly relies on the feedback control subsystem since the command for producing the sound is inaccurate and produces hearing and somatosensory errors. The subsystem detects these errors and sends corrective commands that will be stored by the feedforward system for the next time. In this way, every attempt to produce a sound will on a case-by-case basis be more accurate and will need a lower level of feedback, until the feedforward system is capable of implementing an accurate command for each sound independently, without errors of any kind.

The most accredited hypothesis is that the feedforwrd control system, responsible for motor controls for accurately producing the sound is intact in people who suffer from stuttering. People who suffer from stuttering are conscious of the appropriate and necessary sounds that they intend to produce for the purposes of elocution. On the other hand, if the hearing and somatosensory feedback system does not provide an appropriate contribution, it may generate a command that is not entirely accurate, since the feedforward command is not perfectly aligned with the information coming from the feedback. In these terms, stuttering may reflect an excessive tendency to depend on information coming from the feedback system during the production of language, which tends to send a restart signal to the feedforward control system, which in turn tries to correct the sound by re-starting the current syllable. This gives rise to the need to cultivate the ability to accurately monitor the feedback of one's elocution.

To this end, today electronic apparatuses that are controlled by suitable control units configured to guide or, in general, help a user in practicing vocal exercises are known. Examples of these apparatuses are described in EP0360909 and EP3967223.

### DESCRIPTION OF THE INVENTION

Starting with today's prior art, one purpose of this invention is to propose an innovative vocal practice apparatus, in particular for treating stuttering. In particular, the purpose of this invention is to provide an apparatus that enables the user to improve their ability to monitor feedback understood as greater awareness of how the sound is produced through the control and coordination of all the muscles and parts of the body involved in the planning and production of language (tongue, lips, diaphragm, etc.).

As will become clear, the apparatus of this invention has the goal of re-establishing motor control over the planning and production of the sound in order to more and more accurately align the motor commands for the production of the sound coming out with the information-both hearing and somatosensory-coming in regarding the sound produced. The result that can be obtained thanks to vocal exercises performed using the apparatus of this invention is a reduced influence of the feedback system on the production of the sound owing to a greater monitoring ability and greater independence of the feedforward system through increasingly accurate commands.

According to the most general form of this invention, to achieve the above-mentioned purposes, an innovative vocal practice apparatus is provided, in particular for treating stuttering, wherein the apparatus comprises:
a microphone (which can be over-the-head or of any other form) configured to generate a first audio signal from vocal sounds emitted by a user while performing the exercise;
a camera configured (and obviously positioned so as) to film the user while performing the exercise and to generate a video signal;
a first monitor configured (and obviously positioned so as) to be observed by the user while performing the exercise;
a vibrating device configured to transmit a vibratory impulse to the user while performing the exercise;
a first headset (earbuds or another kind) configured to be worn by the user while performing the exercise.

All the elements mentioned above are individually known and, thus, additional details for correct understanding are not necessary. For detail's sake, the first audio signal is a signal that lasts for the time of performing the exercise and should not be understood as an isolated signal.

There is, finally, a control unit (preferably a PC) connected to the microphone, to the camera, to the first monitor, to the vibrating device, and to the first headset. The connection may be wired or wireless, as the connection may be direct or mediated by other intermediary elements.

In particular, the control unit is configured for:
receiving as input the first audio signal and the first video signal;
generating a second audio signal according to the first audio signal;
transmitting the first audio signal and/or the second audio signal to the first headset so that the user listens to the second audio signal while performing the exercise;
transmitting the video signal to the first monitor so that the user can see himself while performing the exercise;
activating the vibrating device according to the first audio signal and/or the second audio signal during the exercise.

The advantages of performing an exercise of this type are numerous and linked to the coordinated reception of the first and/or second audio signal and of the vibrating stimulus. In fact, the first and the second audio signal, apart from certain exercises, are often simultaneous and the coordination operations are carried out by the control unit. As will be clear, the audio operations can, instead, be managed by an external soundcard, thus not entailing a lag. The control unit preferably provides a wait or delay “timing” for sending the first signal. This timing enables the user to modify the rhythm of fluency. The reception by the user thus makes it possible to improve the feedback mechanisms and internalise the quality of their speech. The vibrating stimulus is synchronised with this timing. This stimulus has the function of modifying the unconscious motor stiffness that the stuttering adds to elocution. The awareness of muscle relaxation during speech helps the user to avoid future muscle spasms. To this end, according to this invention the vibrating stimulus is activated by the control unit during the steps in which the generation of the first signal is not envisaged, i.e. the speech of the patient. This vibratory impulse convinces the brain of the patient not to properly activate muscles, “tricked” by the sensors' doing so. When speech is anticipated, the vibration is interrupted and the patient finds themself in a state of minimal external muscular activation.

In particular, the second audio signal is generated starting from the first audio signal via the following operations (performed by a special application uploaded to the PC that acts as the control unit):
analysis of the first audio signal to identify the features of the voice in terms of frequency and amplitude;
creation of a benchmark of the voice for analysing its departures from the first audio signal compared to the starting condition;
associating the first audio signal with a timing system;
modifying the first audio signal through alteration systems (for example, Altered Auditory Feedback-AAF) with changes to lag times, changes to frequency, or with masking effects.

As mentioned earlier, in addition, the apparatus preferably comprises a soundcard configured to receive the first audio signal, transmit the first audio signal to the control unit, receive the second signal from the control unit, and transmit the second signal to the first headset. In this way, the management of the operations concerning the audio are not the work of the PC's CPU.

The apparatus also preferably comprises a wireless transmitter coupled to the microphone to transmit the first audio signal to the soundcard or directly to the control unit.

In addition, the apparatus may preferably comprise a receiver coupled to the soundcard or to the control unit configured to receive the first audio signal emitted by the wireless transmitter of the microphone.

In addition, the apparatus may preferably comprise a microcontroller coupled to the control unit and configured to control the vibrating device.

In addition, the apparatus may preferably comprise a first wireless receiver coupled to the first headset configured to receive the second audio signal.

In addition, the apparatus may preferably comprise a first wireless transmitter connected to the soundcard and configured to transmit the second audio signal to the wireless receiver coupled to the first headset.

In addition, the apparatus may preferably comprise a second headset, a second wireless receiver coupled to the second headset configured to receive the second audio signal, and a second wireless transmitter connected to the soundcard and configured to transmit the second audio signal to the second wireless receiver coupled to the second headset. In this way, the second audio signal may also be transmitted to another person in addition to the user who is performing the exercise, preferably to a clinician who is monitoring (in person or remotely) the progress of the exercise.

In addition, the apparatus may preferably comprise a second monitor connected to the control unit configured to show information as a function of the first or second audio signal. This information can be viewed by a clinician who monitors (in person or remotely) the progress of the exercise.

In addition, the apparatus comprises a plurality of vibrating devices integrated into a garment that the user can wear during the performance of the exercise. This garment is preferably a jacket or coat in which vibrating devices are integrated in the chest area and in the lower front and rear abdominal area. Still more preferably, this invention comprises sensors, integrated into the garment or which can be worn independently, able to monitor parameters of the patient and transmit these parameters to the control unit in such as way that the apparatus is able to “predict” the onset of the stuttering event and provide the patient with vibrating stimuli to avoid this occurrence. The parameters measured for this purpose that may be predictive of a stuttering event may be sound ones linked to the voice (“voicefeedback”) or physiological (“biofeedback”). For example, the sensors designated for that end are sensors for measuring heart beat, electromyography, skin conductance, skin temperature, and respiration.

In addition, the apparatus may preferably comprise a keyboard or, in general, means that can be controlled by a clinician to set different parameters for performing the exercise i.e. different parameters for generating the second audio signal. The clinician inserts the user's data in the system (through a second monitor, if included). These data will collect a form with the results of the individual tests and feedback in relation to the progress of the whole exercise session. The clinician can also modify (both before and during the exercise) the timing parameters, can alter the frequency of speech, the delay between the first and second signal, and the levels of audio reception and transmission of the first and second signal thus affecting the masking effect as well. These parameters may also be modified while performing the exercise independently by the control unit in certain conditions or they may be modified by the clinician. The control unit has, in fact, a test function: the clinician can decide to record the values of an incoming audio signal of the user to create a reference (benchmark) to be followed in future exercises. This reference is printed at the end of the exercise on the user's form. The clinician can also modify the type of stimulus (visual, hearing, and motor) to be sent to the user, the difficulty of the exercise, and the timing parameters.

The control unit can preferably control the first monitor to show the user some helpful information for performing the exercise or monitoring how the exercise is going.

The apparatus may also be housed or integrated in a structure that comprises a platform, possibly equipped with indications as to where the user must be positioned during the exercise, a vertical support (preferably a cabinet) that supports the first monitor and camera at the front and that houses the PC in a lower position in a compartment.

The vertical support can preferably laterally support the second monitor and the keyboard of the clinician. The platform may also be equipped with a seat and a power socket to power the vibrating device.

Other forms of structures may also, of course, be included. For example, the apparatus may assume a reduced shape in which just one monitor is used, a platform with smaller dimensions, and a proportionally smaller structure.

From a temporal perspective, performing the exercise using the apparatus involves the following sequence of steps:
1. The clinician inserts the data of the user in the system so that, at the end of the exercise, a final form can be generated with the results of the exercise compared with the expected results;

2. The clinician chooses the type of exercise, its difficulty, and other parameters relating to the timing and vibration connected to it;
3. The clinician starts the exercise session in which, in general, the user emits the first signal and receives the first or second signal and the vibrating stimulus as controlled (timing) by the PC;
4. The clinician first asks the user a phrase to record the average amplitude and frequency values that characterise the benchmark;
5. Depending on the exercise, the clinician starts, on a case-by-case basis, the timing; the user practices, receiving feedback from the control unit at each test;
6. In another mode, the user can speak freely, paying attention to the “ranges” provided by the control unit based on benchmark data previously collected;
7. The clinician can decide to record other benchmark values that will influence the following tests;
8. The clinician ends the exercise session and prints the summary form.

Finally, this invention can also be used as a method implemented by a PC for vocal exercises, in particular for the treatment of stuttering. The implementation of the method steps concerns the management of the signals; i.e.:
generating a first audio signal from vocal sounds emitted by a user while performing the exercise;
generating a video signal of the user while performing the exercise;
reproducing the video signal so that the user can see himself while performing the exercise;
transmitting a vibratory impulse to the user while performing the exercise;
generating a second audio signal according to the first audio signal;
transmitting the first and/or the second audio signal to the user so that they listen to it while performing the exercise; in which the vibratory impulse is generated according to the first audio signal and/or the second audio signal while performing the exercise; and in which the generation of the first audio signal and/or transmission of the first and/or second signal to the user may occur with a timing delay. Thus, the protocol of this invention comprises six main steps: 1) acoustic stimulation, 2) visual stimulation, 3) somatosensory stimulation, 4) addition of a non-speech motor act, 5) parameter evaluation, 6) exposure plan for generalisation. These include the three main protocols: acoustic, visual, and somatosensory stimulation.

During the application of the acoustic stimulation protocol, patients are asked to wear headsets connected to the system that replays the manipulated voice of the patient. The manipulation occurs through a specially developed equalisation system that varies the following parameters: frequency, delay, and volume. In particular, the acoustic feedback is not only and simply delayed or altered, but rather it is continuously manipulated, preventing the system from adapting itself. In fact, the acoustic protocol comprises various cycles of increased, exaggerated, and silenced parameters. Patients are asked to concentrate on their vocal production, noting the differences in terms of acoustic qualities during stuttered expressions and those that are not. The final goal is to provide patients with a new acoustic goal: their voice, finally fluent and without blocks and repetitions. During the application of the visual stimulation protocol, patients are asked to synchronise the start of their speech with a visual target provided by the system. The visual stimulation is paired with the equalisation system of the acoustic stimulation. The device displays a visual signal of the right moment that indicates the right moment for starting the speech act to the patient. The patients are asked to observe, in a state of minimal activation, the preparation of the visual cue, thus to start to speak at the exact moment in which the visual cue is provided. Visual feedback on performance is also provided. The purpose of this stimulation is to provide external timing. The external timing is not acoustic (for example, a metronome), but visual. This external visual cue ensures the creation of a new external temporal reference for language. Once the patients are able to focus on the external timing, they are invited to focus on their global (reduced) activation during speech. Therefore, the final goal of this step is to create a new order, characterised by a new, correct timing. During the application of the visual somatosensory protocol, patients are asked to wear the jacket that delivers sensory vibrations that provide vibrations in various areas of the body (for example, the chest and lower abdomen area, both front and rear). In this protocol too, the stimulation is exaggerated with the goal of reproducing the maladaptive hyperactivation of patients'bodies during stuttering. Following this, the somatosensory stimulation, which is paired with the visual one, is reduced when the external view. A clue is provided for timing to enable patients to link the speech act to a state of minimal activation. The final goal is to create a new goal of minimal body activation. The stimulation protocols are then paired with a simple, non-speech motor act. In particular, what has been experimented with before as far as regards the speech act, i.e. the possibility of preparing and producing speech in a fluent way (i.e. without excessive force), is here paired with a small movement of the body that is performed naturally and fluently, without any excessive effort. For example, the arm of the patient that remains still in the initial position and then falls towards gravity, fully resembles a non-stuttering speech, which is performed without excessive activation of the system. In our intervention, this small movement, chosen together with the patient, is then paired with the speech act. It should be noted that the movement in which the arm touches the body of the patient represents the external timing (proprioceptive) to start speech. List of drawings

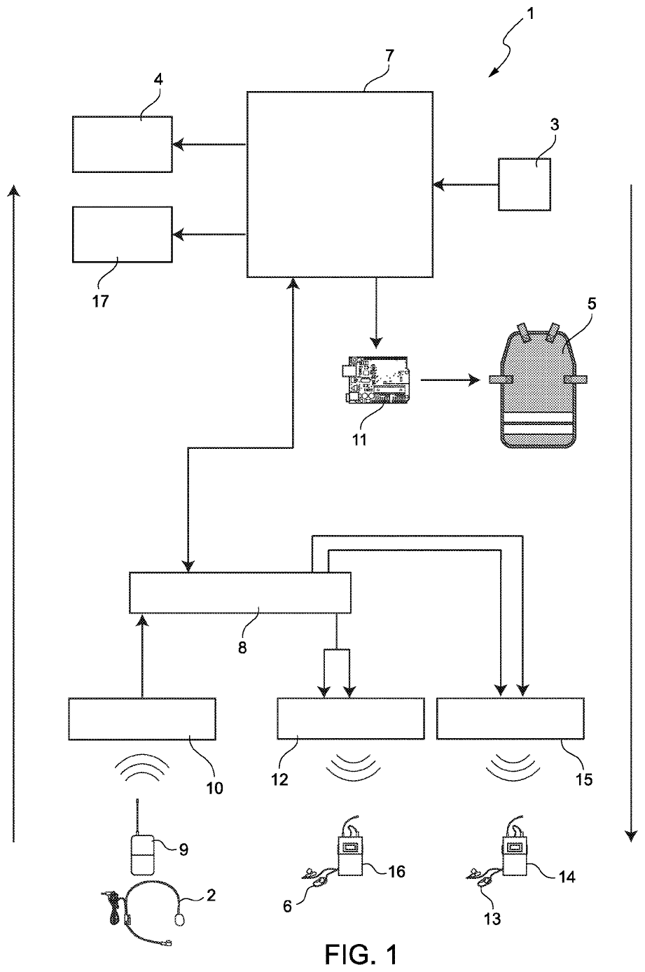

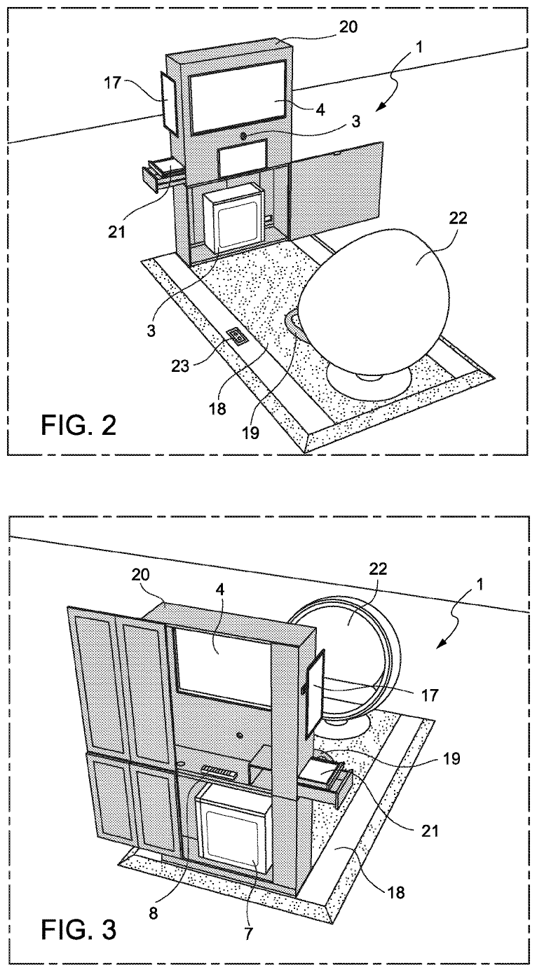

Additional features and advantages of this invention will be clear from the description that follows of a non-limiting embodiment, with reference to the attached figures, in which:
FIG. 1 is a schematic view of the elements and of their interaction within an embodiment of an apparatus according to this invention;
FIGS. 2 and 3 show schematic views of an apparatus of this invention integrated into a support structure.

### DESCRIPTION OF AN EMBODIMENT OF THE INVENTION

Referring to the appended figures, FIG. 1 shows a schematic view of the elements and of their interaction within an embodiment of an apparatus according to this invention. This example is not limiting and some elements are not necessarily always present and they may be produced in different ways as defined by the attached claims. FIG. 1 shows an apparatus indicated, overall, with the reference number 1 and comprises an over-the-head microphone 2 configured to generate a first audio signal from vocal sounds emitted by a user while performing the exercise; this microphone is coupled to a wireless transmitter 9 that transmits the first audio signal to a soundcard 8. This soundcard 8, in turn, transmits the first audio signal to a PC 7 that, starting with this first audio signal, generates a second audio signal that is transmitted to the soundcard 8. This second audio signal is then transmitted to a first headset 6 configured to be worn by the user while performing the exercise by means of a wireless transmitter 12 that collaborates with a corresponding receiver 16 connected to the headset 6. FIG. 1 also shows a second headset 13, a second wireless receiver 14 coupled to the second headset 6 configured to receive the second audio signal, and a second wireless transmitter 15 connected to the soundcard 8 and configured to transmit the second audio signal to the second wireless receiver 16 coupled to the second headset 6. Thus, another person can also listen to the second audio signal. The apparatus 1 in FIG. 1 also comprises a camera 3 configured to film the user while performing the exercise and to generate a video signal, a first monitor 4 configured to be observed by the user while performing the exercise, and multiple vibrating devices integrated into a jacket garment worn by the user while performing the exercise. As shown, the PC 7 collaborates with the elements of the system for
receiving as input the first audio signal and the first video signal;
generating a second audio signal according to the first audio signal;
transmitting the second audio signal to the first headset 6 so that the user listens to the second audio signal while performing the exercise;
transmitting the video signal to the first monitor 4 so that the user can see himself while performing the exercise;
activating the vibrating device 5 according to the first audio signal and/or the second audio signal during the exercise.

Finally, the reference number 11 indicates a microcontroller coupled to the control unit 7 and configured to control the vibrating device 5 via “pwm” (pulse width modulation) signals and the reference number 17 a second monitor 17 connected to the control unit 7 configured to show the clinician information depending on the first or second audio signal.

FIGS. 2 and 3 show schematic views of an apparatus of this invention integrated into a support structure. In this example, the apparatus 1 is housed or integrated in a structure that comprises a platform 18, equipped with indications 19 as to where the user must be positioned during the exercise, a vertical cabinet support 20 (with compartments that can be opened) that supports the first monitor 4 and camera 3 at the front and that houses the PC 7 in a lower position in a compartment. The vertical cabinet support 20 laterally supports the second monitor 17 and a keyboard 21 of the clinician. The platform 18 may also be equipped with a seat 22 and a power socket 23 to power the vibrating device 5.

It is clear that modifications may be made to the invention described herein, and variants produced thereto, in relation to the example shown in the figures.

1. A vocal practice apparatus, in particular for the treatment of stuttering; wherein the apparatus (1) comprises:
a microphone (2) configured to generate a first audio signal from vocal sounds emitted by a user while performing the exercise;
a camera (3) configured to film the user while performing the exercise and to generate a video signal;
a first monitor (4) configured to be observed by the user while performing the exercise;
a plurality of vibrating devices (5) configured to transmit a vibratory impulse to the user while performing the exercise;
a first headset (6) configured to be worn by the user while performing the exercise;
a control unit (7) connected to the microphone (2), the camera (3), the first monitor (4), the vibrating device (5) and the first headset (6);
wherein the control unit (7) is configured for:
receiving as input the first audio signal and the video signal;
generating a second audio signal according to the first audio signal;
transmitting the second audio signal to the first headset (6) so that the user listens to the second audio signal while performing the exercise;
transmitting the video signal to the first monitor (4) so that the user can see himself while performing the exercise;
activating the vibrating device (5) according to the first audio signal and/or the second audio signal during the exercise;
wherein the control unit (7) is configured for activating the vibrating devices (5) with a timing delay with respect to the first audio signal;
wherein there are provided a plurality of vibrating devices (5) integrated into a garment wearable by the user during the performance of the exercise.

2. Apparatus as claimed in claim 1, wherein the control unit (7) is configured for activating the vibrating devices (5) with a timing delay with respect to the first audio signal so that the vibrating devices (5) are activated only during the exercise pauses of no generation of the first audio signal.

3. Apparatus as claimed in claim 1, wherein the garment wearable provided with a plurality of vibrating devices is of the jacket type; the vibrating devices being integrated in the chest area and in the front and rear lower abdominal area.

4. Apparatus as claimed in claim 1, wherein the apparatus comprises sensors suitable for of monitoring physiological parameters of the user and for transmitting these parameters to the control unit; the control unit being configured for activating the vibrating devices depending on these parameters so that to avoid the stuttering generation by activating the vibrating devices.

5. Apparatus as claimed in claim 4, wherein the sensors for monitoring the physiological parameters of the user are sensors for measuring heart rate, and/or electromyography, and/or skin conductance, and/or skin temperature and/or respiration rate.

6. Apparatus as claimed in claim 4, wherein sensors for monitoring the physiological parameters of the user are integrated into the garment wearable by the user during the performance of the exercise provided with the vibrating devices.

7. Apparatus as claimed in claim 1 wherein the apparatus further comprises a soundcard (8) configured to receive the first audio signal, transmit the first audio signal to the control unit (7), receive the second signal from the control unit (7), and transmit the first and/or second signal to the first headset (6).

8. Apparatus as claimed in claim 1, wherein the apparatus further comprises a wireless transmitter (9) coupled to the microphone for transmitting the first audio signal to the soundcard (8) or directly to the control unit (7).

9. Apparatus as claimed in claim 8, wherein the apparatus further comprises a receiver (10) coupled to the soundcard (8) or control unit (7) configured to receive the first audio signal output from the wireless transmitter (9).

10. Apparatus as claimed in claim 1, wherein the apparatus further comprises a microcontroller (11) coupled to the control unit (7) and configured to control the vibrating device (5).

11. Apparatus as claimed in claim 1, wherein the apparatus further comprises a first wireless receiver (16) coupled to the first headset (6) configured to receive the second audio signal.

12. Apparatus as claimed in claim 11, wherein the apparatus further comprises first wireless transmitter (12) connected to the soundcard (8) and configured to transmit the second audio signal to the wireless receiver (16) coupled to the first headset (6).

13. Apparatus as claimed in claim 11, wherein the apparatus further comprises a second headset (13), a second wireless receiver (14) coupled to the second headset (6) configured to receive the second audio signal, and a second wireless transmitter (15) connected to the soundcard (8) and configured to transmit the second audio signal to the second wireless receiver (16) coupled to the second headset (6).

14. Apparatus as claimed in claim 1, wherein the apparatus further comprises a second monitor (17) connected to the control unit (7) configured to display information function of the first or second audio signal.
